(19) United States
(12) Patent Application Publication
(10) Pub. No.: US 2005/0106223 A1
(43) Pub. Date: May 19, 2005

(54) Multilayer personal cleansing and/or moisturizing article

(76) Inventor: Albert R. Kelly, Douglaston, NY (US)

Correspondence Address: EVELYN M. SOMMER, 250 PARK AVE, RM 825, NEW YORK, NY, 10022, US

(21) Appl. No.: 10/879,455

(22) Filed: Jun. 30, 2004

Related U.S. Application Data
(60) Provisional application No. 60/519,584, filed on Nov. 14, 2003.

(51) Int. Cl. A61K 9/70

(52) U.S. Cl. 424/443

(57) ABSTRACT

A personal skin cleansing and/or moisturizing article comprising a trilaminate sonically bonded or needle punched structure impregnated with a cleansing and/or moisturizing emulsion that is air dried to remove all of the free water, and upon saturation with water and the application of pressure and friction in use, the original emulsion is instantly reconstituted.

### FIELD OF INVENTION

The present invention relates to a personal skin cleansing and/or moisturizing wipe comprising a trilaminate sonically or needle punch bonded substrate which has been impregnated with a liquid cleansing and/or moisturizing phase-inversion temperature emulsion. The article may be provided as a wipe, flat sheet, flat folded, or cross-folded, or as a pillow, facemask or in the form of a mitt.

### BACKGROUND OF THE INVENTION

Wipes for both body and skin cleansing are known and have been described for example in U.S. Pat. Nos. 5,980,931, 6,063,397, and 6,074,655, which teach a substantially dry disposable personal cleansing product useful for both cleansing and conditioning the skin. A disposable cleansing product having a substrate comprising multiple layers is taught in U.S. Pat. No. 6,060,149.

U.S. Pat. Nos. 6,063,746, 6,537,562, 6,576,678 B1, 6,410,039 B1, 5,817,254 and others teach cleansing compositions for incorporation into wipe products. U.S. Pat. Nos. 6,440,437 B1 and 6,410,039 B1 teaches the inclusion of an oil-in-water emulsion into a soft wet wipe or wipe-type products. U.S. Pat. Nos. 5,993,832 and 6,562,876 B1 also teach oil-in-water emulsions which can be used in cosmetics.

U.S. Pat. No. 6,537,562 B1 teaches cosmetic phase-inversion temperature (PIT) emulsions for use in refatting systems having good performance characteristics and high stability in storage. U.S. Pat. No. 6,623,746 B1 teaches a PIT emulsion for softening substrates, such as papers, nonwovens, tissues and cloths.

### SUMMARY OF THE INVENTION

A personal skin cleansing and/or moisturizing article comprising a water insoluble trilaminate substrate comprising, 1) a top layer composed of at least in part synthetic fibers, preferably polypropylene or polypropylene and rayon fibers, 2) a highly absorbent, highly stretchable and lightweight cellulose core and 3) a bottom layer composed of substantially synthetic fibers, preferably thermally or spun bonded polypropylene, wherein the top layer, center layer and bottom layer are sonically or needle punch bonded together, impregnated with a cleansing and/or moisturizing emulsion containing:
(a) C₈₋₂₂ and preferably C₁₂₋₁₈ fatty acid alkyl esters,
(b) C₈₋₂₂ and preferably C₁₂₋₁₈ fatty alcohols,
(c) C₈₋₂₂ and preferably C₁₂₋₁₈ alcohol polyglycol ethers,
(d) C₈₋₂₂ and preferably C₁₂₋₁₈ fatty acid partial glycerides, and optionally
(e) auxiliaries and additives

As used herein the term “article” is intended to cover the various product forms such as flat sheets, folded sheets, cushions, masks or mitts.

### DETAILED DESCRIPTION OF THE INVENTION

The present invention provides personal skin cleansing and/or moisturizing articles of the non-wet type, which have a particularly soft feel, and excellent cleaning and moisturizing properties, useful for both cleansing and conditioning the skin, comprising,
(a) a water insoluble substrate comprising,
   (1) a first water insoluble nonwoven layer comprised of at least in part synthetic fibers, preferably polypropylene or polypropylene and rayon fibers, having outer and inner surfaces,
   (2) a water insoluble core layer made of cellulosic wadding having a density less than 0.3 and preferably less than 0.2 grams per cubic centimeter and most preferably in the range of 0.01 to 0.05 grams per cubic centimeter, having a stretch capacity of at least 30%, having a water absorbent capacity of at least 12, preferably up to 17 and most preferably 12-15 grams per gram of its basis weight, and,
   (3) a second water insoluble nonwoven layer comprised substantially of synthetic fibers, preferably thermally or spun bonded polypropylene, having outer and inner surfaces wherein the core layer is positioned between the inner surfaces of said first and second layers and the first, second layers and intermediate core layer are through bonded by sonic bonding to form a unified article having perforations of less than 0.5 mm in diameter through all of said layers, said perforations having been formed during said sonic bonding. Alternatively, the first, second layers and intermediate core layer are mechanically through-bonded by needling to form a unified article having perforations of less than 0.3 mm in diameter extending through all of said layers, said perforations having been formed during said mechanical bonding. In either embodiment, said perforations assist in controlling water absorption into the core required for activating the cleansing solution and for the controlled and repeated release of the moisturizing solution upon its activation by said absorbed water.
(b) an amount of a liquid, cleansing and/or moisturizing emulsion being impregnated in said water insoluble substrate to deliver 6 to 24 grams of emulsion in use, wherein said liquid cleansing and/or moisturizing composition comprises,
   (1) C₈₋₂₂ and preferably C₁₂₋₁₈ fatty acid alkyl esters,
   (2) C₈₋₂₂ and preferably C₁₂₋₁₈ fatty alcohols,
   (3) C₈₋₂₂ and preferably C₁₂₋₁₈ alcohol polyglycol ethers,
   (4) C₈₋₂₂ and preferably C₁₂₋₁₈ fatty acid partial glycerides and,
   (5) Optionally auxiliaries and additives.
and most advantageously the compositions comprise,
   (1) 1 to 70%, preferably 30 to 50% by weight of C₈₋₂₂ fatty acid alkyl esters,
   (2) 1 to 40%, preferably 10 to 20% by weight of C₈₋₂₂ fatty alcohols,
   (3) 1 to 40%, preferably 10 to 20% by weight of C₈₋₂₂ alcohol polyglycol ethers,
   (4) 1 to 40%, preferably 10 to 20% by weight of C₈₋₂₂ fatty acid partial glycerides and optionally
   (5) 0 to 70%, preferably 10 to 50% by weight of auxiliaries and additives, with the proviso that the quantities add up to 100% by weight.

The water insoluble substrates are preferably composite materials as described in copending applications Ser. No. 10/021,395 and Ser. No. 10/222,401. The top layer can be 100% polypropylene or a mixture of polypropylene and rayon. The polypropylene layer can range from about 1.0 to about 3.5 oz/sq. yd. The bottom layer is a 0.5 to 2 oz/sq. yd. of a thermally or spun bonded polypropylene.

The top and bottom layers can also be comprised of a mixture of the fibers having high and low deniers incorporated therein. These are added in order to form a rough or exfoliating surface on at least the face of the article. An outer layer can also be embossed or debossed ultrasonically so that it exhibits a pattern of raised and depressed areas or surface aberrations as an alternative to including abrasive particles or flame treating and the like. This abrasive or embossed surface also serves to enhance the cleaning or exfoliating effect.

The cellulosic materials suitable for use in forming the core layer are fibers composed or derived from cellulose. Examples are paper (cellulose), hemp and cotton. Particularly preferred fibers are derived from wood pulp. The cellulosic core can be a wadding or other like cellulosic material.

The desired density of the core material is less than 0.3 grams per cubic centimeter and preferably no more than 0.2 grams per cubic centimeter and most preferably in the range of 0.01 to 0.05 grams per cubic centimeter. The core material can absorb up to 17 grams per gram of its basis weight, preferably 12 to 15 grams per gram of its basis weight. As a comparison, conventional papers have densities well in excess of 0.3 grams per cubic centimeter, for example, 0.5 to 0.7 grams per cubic centimeter, and an absorbency of about 4 to 7 grams per gram of their basis weight. Because it is essential for the core to retain a relatively large amount of treatment composition, generally more than 6 grams per wipe, to allow for repeated use, the use of a low absorbency cores yield wipes which become nearly completely or over saturated, when the required amount of treatment approaches or exceeds the absorption capacity of the core. Thus conventional paper cores, with an absorbent capacity of only up to 7 grams per gram of its basis weight, will not yield a wiper product that can retain and gradually release more than 6 grams of composition needed to be effective. Attempting to treat a 7″×8″ wiper product having a core with a basis weight of 28 lb/ream with a 6 gram add-on would result in nearly complete or over saturation of the core. In that event, excess of the composition will leave a glossy hand feel of the dried product. This can also give rise to more rapid activation and release of the treatment composition and a reduced span of use.

Additionally, for the cellulosic core to survive the multidirectional tensions incurred during bonding, under high speed converting conditions and similar tensions incurred in use, the core must possess high stretch capacity, at least 30% and preferably up to 75%, which is well in excess of the 1% to 2% commonly found with conventional paper cores. If conventional paper cores are used, their function as a wipe, including their use and potential for repeated use will be severely compromised.

The preferred core is a cellulosic wadding core with a basis weight of 22 lb/ream to 35 lb/ream, preferably 28 lb/ream, that has an unusually high stretch capacity of about 75%, and a low density of less than 0.2 grams per cubic centimeter, preferably less than 0.1 grams per cubic centimeter. Shawano Specialty Papers produce cellulosic wadding of this type as Grade 1804. These characteristics allow the core to survive needling and preserve structural integrity of the composite structure under the multidirectional tensions during converting and in use. The high absorption capacity of up to 17 grams of water per gram of basis weight, or 27.2 grams of water per 7″×8″ wipe, is also critical in allowing the core to absorb and retain 6 grams of cleaning composition (representing about 22% of its capacity) without risking treatment composition migration, or its premature release.

By comparison, standard papers have densities well in excess of 0.3 grams per cubic centimeter, absorb only up to 7 grams per gram of their basis weight and can stretch only by about 1.5%. The use of conventional paper results in a wipe product that cannot retain and gradually release the high load of treatment composition required for products of this type to be effective.

The product of the present invention comprising multiple layers may be ultrasonically bonded after applying the coating of one or more of the layers. Alternatively layers may be bonded together by needle punch, or sonic bonding prior to applying the coating.

The wipes contain PIT emulsions as impregnating formulations. The emulsions produced by the phase inversion temperature process have low viscosities even in highly concentrated form so that they are easy to process. By virtue of their small droplet size (<100 μm), the emulsions penetrate very quickly into the core and are uniformly dispersed therein.

The PIT emulsions contain,
(1) C₈₋₂₂ and preferably C₁₂₋₁₈ fatty acid alkyl esters,
(2) C₈₋₂₂ and preferably C₁₂₋₁₈ fatty alcohols,
(3) C₈₋₂₂ and preferably C₁₂₋₁₈ alcohol polyglycol ethers and,
(4) C₈₋₂₂ and preferably C₁₂₋₁₈ fatty acid partial glycerides.

In particularly preferred embodiments the emulsions contain,
(1) 1 to 70%, preferably 30 to 50% by weight of fatty acid alkyl esters,
(2) 1 to 40%, preferably 10 to 20% by weight of fatty alcohols,
(3) 1 to 40%, preferably 10 to 20% by weight of alcohol polyglycol ethers,
(4) 1 to 40%, preferably 10 to 20% by weight of fatty acid partial glycerides,
(5) 0 to 70%, preferably 10 to 50% by weight of auxiliaries and additives,
   with the proviso that the quantities add up to 100% by weight.

The cleansing composition of this invention may, if desired, also contain components either to provide additional effect or to make the product more attractive to the consumer. The following are mentioned by way of example: antibacterial agents such as 2,4,4′-trichloro-2′-hydroxydiphenyl ether colors or dyes in amounts up to 0.5% by weight; preservatives or antioxidizing agents, such as formalin, 5-bromo-5-nitro-1,3-dioxane; 5-chloro-2-methyl-4-isothaliazolin-3-one, 2,6-di-tertbutyl-p-cresol, etc., in amounts up to 2% by weight; and pH adjusting agents, such as sulfuric acid or sodium hydroxide, as needed.

The emulsions are prepared as concentrates with an active substance content of 10 to 70% by weight and are subsequently diluted to an end use concentration of about 1 to about 15% by weight. If desired, the aqueous phase may also contain polyols, preferably up to 15% by weight of glycerol.

The treatment of the trilaminate with the cleansing and/or moisturizing preparations is carried out with the solution being applied to at lest one side of the laminate or composite structure. Basically, any known method by which liquids or melts can be applied to flexible substrate products may be used for this purpose, including for example spraying, printing (for example flexography printing), coating (gravure coating), extrusion spot coating and combinations of these methods. In the alternative, the core may be impregnated with the formulations prior to being laminated.

The water is present in the composition at a concentration of about 50 weight % to 70 weight %. The trilaminate structure is impregnated with the cleansing and/or moisturizing emulsion so that it contains 6 to 24, preferably 10 to 20 grams of the emulsion. Following impregnation, the impregnated substrates are subjected to air drying to substantially remove all free water. In use, on saturation with water and application of pressure, the original emulsion is quickly reconstituted.

It has been observed that when PIT emulsions as herein employed are used to impregnate other substrates than those specifically identified herein and then subjected to air drying so that substantially all of the water present in the emulsion is removed, this treatment step results in the quick and efficient reconstitution of the original emulsion when the article is wet with water and some pressure applied.

The following examples illustrate the cleansing and/or moisturizing wipes of the described invention. Unless otherwise specified, all percentages are by weight. The exemplified compositions are illustrative only and do not limit the scope of the invention.

### EXAMPLE 1

A sonically bonded trilaminate substrate having the following structure was prepared as described in copending application Ser. No. 10/107,901:
a) 1 oz polypropylene,
b) 34 lb wadding (creped paper),
c) 1 oz thermally or spun bonded polypropylene.

The core (b) had a density of 0.05 grams per cubic centimeter. The laminate was impregnated with an emulsion having the following composition
a) cetyl palmitate 30% by wt
b) hydrogenated castor oil 4% by wt
c) glycerol stearate 2% by wt
d) beheneth 10% by wt
e) water to 100%

The impregnated laminate is air dried to remove all free water. On activation by saturation with water, the emulsion is reformed and released slowly.

### EXAMPLE 2

A needle punch bonded trilaminate having the following structure,
a) 2.5 oz polypropylene and rayon,
b) 34 lb wadding (creped paper),
c) 1.0 oz thermally or spun bonded polypropylene
   was prepared as described in copending application Ser. No. 10/222401. The core (b) had a density of 0.05 grams per cubic centimeter. It was impregnated with an emulsion having the following composition,
a) cetyl palmitate 40% by wt
b) hydrogenated castor oil 6% by wt
c) glycerol stearate 3% by wt
d) beheneth 10% by wt
e) water to 100%

The impregnated laminate was air dried to remove all free water.

The impregnate was used in an amount calculated to deliver 20 grams of the emulsion on being activated by saturation of the wipe with water.

### EXAMPLE 3 and 4

Examples 1 and 2 were repeated but for the third or bottom layer 0.5 oz thermally or spun bonded polypropylene was used. The same results were achieved.

1. A disposable flexible article useful for personal skin cleansing and/or moisturizing comprising a laminate comprising:
a) a first water insoluble nonwoven layer comprised at least in part synthetic fibers, and having outer and inner surfaces,
b) a water insoluble core layer made of cellulosic wadding having a density less than 0.3 and preferably less than 0.2 grams per cubic centimeter, having a stretch capacity of at least 30%, having a water absorbent capacity of at least 12 grams per gram of its basis weight, incorporating about 25% to about 300% of the article's total basis weight of a treatment composition adapted for personal skin cleansing or moisturizing wherein said treatment composition comprises,
1) C₈₋₂₂ fatty acid alkyl esters,
2) C₈₋₂₂ fatty alcohols,
3) C₈₋₂₂ alcohol polyethers,
4) C₈₋₂₂ fatty acid partial glycerides and
c) a second water insoluble nonwoven layer comprised substantially of synthetic fibers having outer and inner surfaces wherein the core layer is positioned between the inner surfaces of said first and second layers and the first, second and intermediate core layer are through-bonded by sonic bonding to form a unified article having perforations of less than 0.5 mm in diameter through all of said layers, said perforations having been formed during said sonic bonding, wherein said perforations assist in controlling water absorption into the core required for activating the cleaning solution and for the controlled and repeated release of the cleaning solution upon its activation by said absorbed water.

2. A disposable flexible article useful for personal skin cleansing and/or moisturizing comprising a laminate comprising:
a) a first water insoluble nonwoven layer comprised at least in part synthetic fibers, and having outer and inner surfaces,
b) a water insoluble core layer made of cellulosic wadding having a density less than 0.3 and preferably less than 0.2 grams per cubic centimeter, having a stretch capacity of at least 30%, having a water absorbent capacity of at least 12 grams per gram of its basis weight, incorporating about 25% to about 300% of the article's total basis weight of a treatment composition adapted for personal skin cleansing or moisturizing wherein said treatment composition comprises,
1) C₈₋₂₂ fatty acid alkyl esters,
2) C₈₋₂₂ fatty alcohols,
3) C₈₋₂₂ alcohol polyethers,
4) C₈₋₂₂ fatty acid partial glycerides
5) optionally adjuvant additives and
c) a second water insoluble nonwoven layer comprised substantially of synthetic fibers having outer and inner surfaces wherein the core layer is positioned between the inner surfaces of said first and second layers and the first, second and intermediate core layer are mechanically through-bonded by needling to form a unified article having perforations of less than 0.3 mm in diameter extending through all of said layers, said perforations having been formed during said mechanical bonding, wherein said perforations assist in controlling water absorption into the core required for activating the cleaning solution and for the controlled and repeated release of the cleaning solution upon its activation by said absorbed water.

3. A disposable flexible article useful for personal skin cleansing and/or moisturizing according to claim 1, wherein said first layer is composed of polypropylene.

4. A disposable flexible article useful for personal skin cleansing and/or moisturizing according to claim 2, wherein said first layer is composed of polypropylene.

5. A disposable flexible article useful for personal skin cleansing and/or moisturizing according to claim 1, wherein said first layer is composed of polypropylene and rayon.

6. A disposable flexible article useful for personal skin cleansing and/or moisturizing according to claim 2, wherein said first layer is composed of polypropylene and rayon.

7. A disposable flexible article useful for personal skin cleansing and/or moisturizing according to claim 1, wherein said second layer is composed of polypropylene.

8. A disposable flexible article useful for personal skin cleansing and/or moisturizing according to claim 2, wherein said second layer is composed of polypropylene.

9. A disposable flexible article according to claim 1, wherein the said C₈₋₂₂ fatty acid alkyl esters are present at 1 to 70% by weight, the said C₈₋₂₂ fatty alcohols are present at 1 to 40% by weight, the said C₈₋₂₂ alcohol polyethers are present at 1 to 40% by weight, the said C₈₋₂₂ fatty acid partial glycerides are present at 1 to 40% by weight, and the said adjuvant additives are present at 0 to 70% by weight.

10. A disposable flexible article according to claim 2, wherein the said C₈₋₂₂ fatty acid alkyl esters are present at 1 to 70% by weight, the said C₈₋₂₂ fatty alcohols are present at 1 to 40% by weight, the said C₈₋₂₂ alcohol polyethers are present at 1 to 40% by weight, the said C₈₋₂₂ fatty acid partial glycerides are present at 1 to 40% by weight, and the said adjuvant additives are present at 0 to 70% by weight.

11. A disposable flexible article useful for personal skin cleansing and/or moisturizing according to claim 1 wherein said core of laminate prior to impregnation with said treatment composition has a density of less than 0.05 grams per cubic centimeter.

12. A disposable flexible article useful for personal skin cleansing and/or moisturizing according to claim 2 wherein said core of laminate prior to impregnation with said treatment composition has a density of less than 0.05 grams per cubic centimeter.

13. A disposable flexible article useful for personal skin cleansing and/or moisturizing according to claim 1 wherein at least one of the said fatty acid alkyl esters corresponds to the general formula R¹CO—OR², R¹CO representing a linear or branched, saturated or unsaturated acyl group having from 8 to 22 carbon atoms and R² representing a linear or branched, alkyl or alkenyl group having from 6 to 22 carbon atoms; wherein at least one of the fatty alcohols corresponds to the general formula R³OH, R³ representing a linear or branched, alkyl or alkenyl group having from 8 to 22 carbon atoms; wherein at least one of the alcohol polyglycol ethers corresponds to the general formula R⁴O(CH₂CHR⁵O)ₙH, R⁴ representing a linear or branched, alkyl or alkenyl group having from 8 to 22 carbon atoms, R⁵ representing a hydrogen atom or a methyl group, and n being a number of from 1 to 50; wherein at least one fatty acid partial glycerides comprises a monoglyceride component corresponding to the general formula HOCH₂CH(OH)CH₂OCOR⁶, R⁶ representing a linear or branched, saturated or unsaturated acyl group having from 8 to 22 carbon atoms.

14. A disposable flexible article useful for personal skin cleansing and/or moisturizing according to claim 2 wherein at least one of the said fatty acid alkyl esters corresponds to the general formula R¹CO—OR², R¹CO representing a linear or branched, saturated or unsaturated acyl group having from 8 to 22 carbon atoms and R² representing a linear or branched, alkyl or alkenyl group having from 6 to 22 carbon atoms; wherein at least one of the fatty alcohols corresponds to the general formula R³OH, R³ representing a linear or branched, alkyl or alkenyl group having from 8 to 22 carbon atoms; wherein at least one of the alcohol polyglycol ethers corresponds to the general formula R⁴O(CH₂CHR⁵O)ₙH, R⁴ representing a linear or branched, alkyl or alkenyl group having from 8 to 22 carbon atoms, R⁵ representing a hydrogen atom or a methyl group, and n being a number of from 1 to 50; wherein at least one fatty acid partial glycerides comprises a monoglyceride component corresponding to the general formula HOCH₂CH(OH)CH₂OCOR⁶, R⁶ representing a linear or branched, saturated or unsaturated acyl group having from 8 to 22 carbon atoms.

15. Method of manufacturing an article according to claim 1 wherein the said treatment composition is applied to the substrate in an aqueous formulation and the said substrate is air-dried to remove the free water after the application of the treatment composition to the.

16. Method of manufacturing an article according to claim 2 wherein the said treatment composition is applied to the substrate in an aqueous formulation and the said substrate is air-dried to remove the free water after the application of the treatment composition to the article.

17. Method of cleansing skin comprising saturating an article according to claim 1 with water prior to or simultaneously with contact with the skin to be cleaned whereby the impregnant in the article is reformed into the emulsion and is slowly released onto the skin.

18. Method of cleansing skin comprising saturating an article according to claim 2 with water prior to or simultaneously with contact with the skin to be cleaned whereby the impregnant in the article is reformed into the emulsion and is slowly released onto the skin.

19. A flexible disposable article according to claim 1 wherein said article is in the form of a flat sheet, folded sheet, cushion or mitt forms.

20. A flexible disposable article according to claim 2 wherein said article is in the form of a flat sheet, folded sheet, cushion or mitt forms.
